(19) United States
(12) Patent Application Publication
(10) Pub. No.: US 2014/0200632 A1
(43) Pub. Date: Jul. 17, 2014

(54) SELF-DIRECTED REHABILITATION TRAINING METHOD COMBINING BRAIN SIGNALS AND FUNCTIONAL ELECTROSTIMULATION

(71) Applicant: Daegu Gyeongbuk Institute of Science and Technology, Daegu (KR)

(72) Inventors: Jinung AN, Daegu (KR); Sang Hyeon JIN, Daegu (KR); Seung Hyun LEE, Daegu (KR); Jeon Il MOON, Daegu (KR); Berdakh ABIBULLAEV, Astana (KZ); Jae Hyun AHN, Daegu (KR); Gwang Hee JANG, Daegu (KR)

(73) Assignee: Daegu Gyeongbuk Institute of Science and Technology, Daegu (KR)

(21) Appl. No.: 14/049,302

(22) Filed: Oct. 9, 2013

(30) Foreign Application Priority Data
Jan. 11, 2013 (KR) 10-2013-0003585

(51) Int. Cl. A61N 1/36

(52) U.S. Cl. CPC A61N 1/36
USPC 607/62

(57) ABSTRACT

Provided is a self-directed rehabilitation training method for providing rehabilitation exercise to a patient needing requiring the rehabilitation according to a will of the patient, which is a rehabilitation training method for providing rehabilitation intention inducing environment to the patient, measuring the state of the patient, and providing rehabilitation exercise appropriate for the patient. According to the present invention, it is possible for the patient to do the rehabilitation exercise in an active environment by measuring brain signals of the patient to adjust intensity or time of the rehabilitation exercise.

### CROSS-REFERENCE TO RELATED APPLICATIONS

This application claims priority under 35 U.S.C. §119 to Korean Patent Application No. 10-2013-0003585, filed on Jan. 11, 2013, the disclosure of which is incorporated herein by reference in its entirety.

### TECHNICAL FIELD

The present invention relates to a rehabilitation training provision method for providing rehabilitation exercise to a patient having damage on the central nervous system due to brain or vertebra damage, a patient having disease on the musculoskeletal system, or a patient requiring sport rehabilitation, and more particularly, to a self-directed rehabilitation training method using active self-training.

### BACKGROUND

Rehabilitation treatment denotes a series of measure process which is performed for functionally recovering a damaged part or function weakened part of a patient whose body part is damaged by a disease, an accident, a disaster, or the like, or who undergoes severe surgery and then is in a convalescent stage.

Conventional rehabilitation treatment is performed by a therapist, a robot, or an electrical simulator, or the like, and thus is generally provided to patients unilaterally and passively.

For this reason, from the cerebral nerve perspective, since complete sensor-motor looped rehabilitation is not made, the conventional rehabilitation treatment can be considered as a method applicable to acute patients or subacute patients, but is not suitable for chronic patients undergoing a rehabilitation plateau that is a period for which a rehabilitation effect is no longer improved by passive rehabilitation and a current rehabilitation state is maintained as-is.

### SUMMARY

Accordingly, the present invention provides a self-directed rehabilitation training method for presenting the rehabilitation training appropriate for the patient as sensing information such as vision, measuring a bio-signal associated with the central nervous system, musculoskeletal system, or sensory system, estimating a state of a patient, and providing stimulation or excise appropriate for the state in order to enhance neuroplasticity, musculoskeletal resilience, and exercisable range separately or simultaneously.

In one general aspect, a self-directed rehabilitation training method includes: allowing a patient to put a cap having a measurement probe on a scalp of the patient and attaching a patch to a rehabilitation portion of the patient; providing the patient with a rehabilitation operation screen; measuring cerebral cortical blood flow and metabolism of the patient using the cap to generate a measured signal; extracting a feature of the measured signal and classifying rehabilitation intention on the basis of the feature; converting the rehabilitation intention into an instruction for presenting stimulation and providing the patient with rehabilitation exercise according to the instruction for presenting stimulation.

The attaching of a patch to a rehabilitation portion of the patient may further include attaching a patch for electromyogram measurement to a skin portion around the rehabilitation portion.

The providing of the patient with a rehabilitation operation screen may include providing the patient with the rehabilitation operation screen repeatedly for a predetermined time period.

The generating of the measured signal may include measuring an oxyhemoglobin concentration and a deoxyhemoglobin concentration on the basis of the blood flow to generate the measured signal; removing noise from the measured signal through a preprocessing method; and providing the measured signal with the noise removed therefrom when extracting the feature for the rehabilitation intention classification.

The providing of the patient with rehabilitation exercise may include operating the rehabilitation portion of the patient by applying functional electrostimulation to the patient to the maximum extent in terms of intensity and duration according to the instruction for presenting stimulation; measuring a degree of muscle contraction of the patient after applying the functional electrostimulation to the patient, and if the muscle activity exceeds a predetermined value, gradually reducing the intensity and duration of the functional electrostimulation; and providing the muscle activity to the patient through a screen.

Other features and aspects will be apparent from the following detailed description, the drawings, and the claims.

### BRIEF DESCRIPTION OF THE DRAWINGS

FIGS. 1 and 2 are flowcharts each showing a process of a self-directed rehabilitation training method according to an embodiment of the present invention.

FIG. 3 illustrates a brain image showing cerebral cortical blood flow and metabolism measured when providing rehabilitation exercise through the self-directed rehabilitation training method according to an embodiment of the present invention.

FIG. 4 illustrates a signal showing a hemoglobin concentration measured when providing rehabilitation exercise through the self-directed rehabilitation training method according to an embodiment of the present invention.

### DETAILED DESCRIPTION OF EMBODIMENTS

The advantages, features and aspects of the present invention will become apparent from the following description of the embodiments with reference to the accompanying drawings, which is set forth hereinafter. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Hereinafter, embodiments of the present invention will be described in detail with reference to the accompanying drawings.

The present invention supplements conventional rehabilitation training method that provides passive rehabilitation exercise to patients requiring rehabilitation, and provide an active rehabilitation method for inducing rehabilitation intention by allowing a patient to watch rehabilitation operations presented on a screen according to predetermined rehabilitation guidelines or image rehabilitation operations presented by a speaker, or voice of the therapist, measuring cerebral cortical signals of the patient in a noninvasive method with a brain-signal measuring instrument while inducing the rehabilitation intention, extracting the current intention of the patient from the measured cerebral cortical signals, and applying functional electrostimulation appropriate for the intention, thereby enabling self-rehabilitation.

In this case, the rehabilitation motivation of the patient may be activated by presenting digitalized rehabilitation effect, such as a score, to the patient on a screen, and the degree of muscle contraction of the patient may be measured as electromyogram to adjust the intensity of the functional electrostimulation appropriate for the muscle activity based on the rehabilitation.

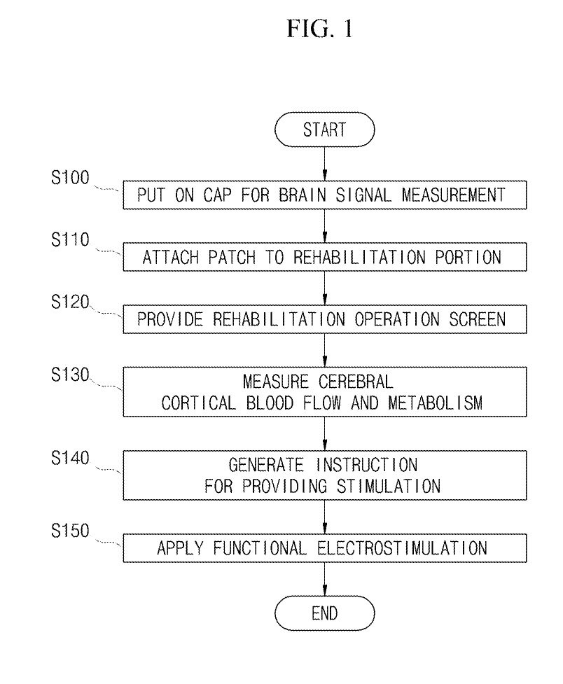

FIG. 1 is a flowchart showing a process of a self-directed rehabilitation training method according to an embodiment of the present invention.

The self-directed rehabilitation training method according to an embodiment of the present invention puts a cap having a measurement probe on a scalp of a patient in order to measure a brain signal (Functional Near Infrared Spectroscopy (fNIRS) or electroencephalogram (EEG)) in operation S100 and finds an appropriate body part for rehabilitation and attaches a patch on skin of the body part to apply functional electrostimulation thereto in operation S110.

After the cap is put on to measure a patient state and the patch is attached to provide rehabilitation exercise, the self-directed rehabilitation training method presents a rehabilitation operation to a patient through a screen while keeping the patient comfortable in operation S120.

The providing of the rehabilitation exercise screen to a patient may be performed by providing the rehabilitation exercise screen to the patient repeatedly for a predetermined time period in consideration of rehabilitation intensity required to the patient and rehabilitation exercise level of the patient. Thus, the patient repeatedly watches or imagines the presented rehabilitation operations for a certain time. Also, when providing the rehabilitation exercise screen to the patient, the rehabilitation exercise may be provided through a speaker or a therapist's voice to stimulate rehabilitation motivation of the patient.

After providing the rehabilitation exercise screen to the patient, the method measures the state of the patient to provide the rehabilitation exercise appropriate for the patient in operation S130, in which the measuring of the patient state may be performed by measuring the cerebral cortical blood flow and metabolism through the cap put on the scalp of the patient.

The measuring of the cerebral cortical blood flow and metabolism of the patient may be performed using Functional Near Infrared Spectroscopy (fNIRS), and the patient state may be measured by measuring electroencepalogram (EEG) through potential variation of the cerebrum.

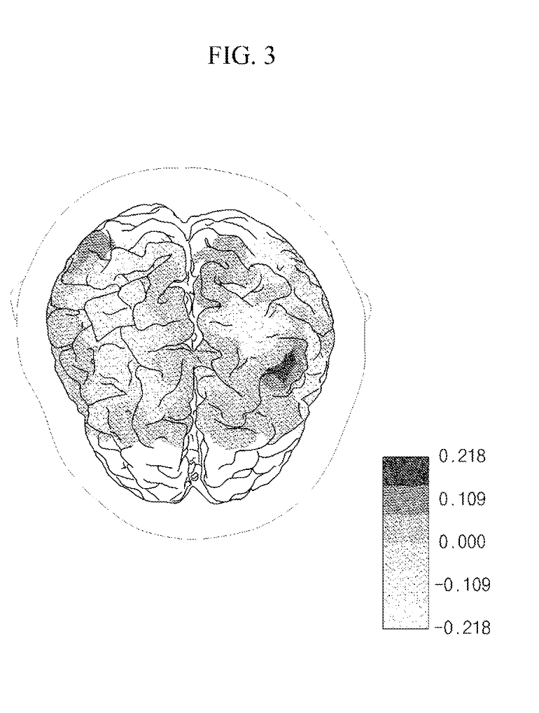

When measuring the cerebral cortical blood flow and metabolism, as shown in FIG. 3, the cerebral cortical blood flow and metabolism may be monitored in a form of a brain image where the activity of the brain is represented by color and number. This allows the patient to check the brain activity with his/her eye while the patient undertakes rehabilitation training. As the number is great in the brain image, the frequency of the activity is high.

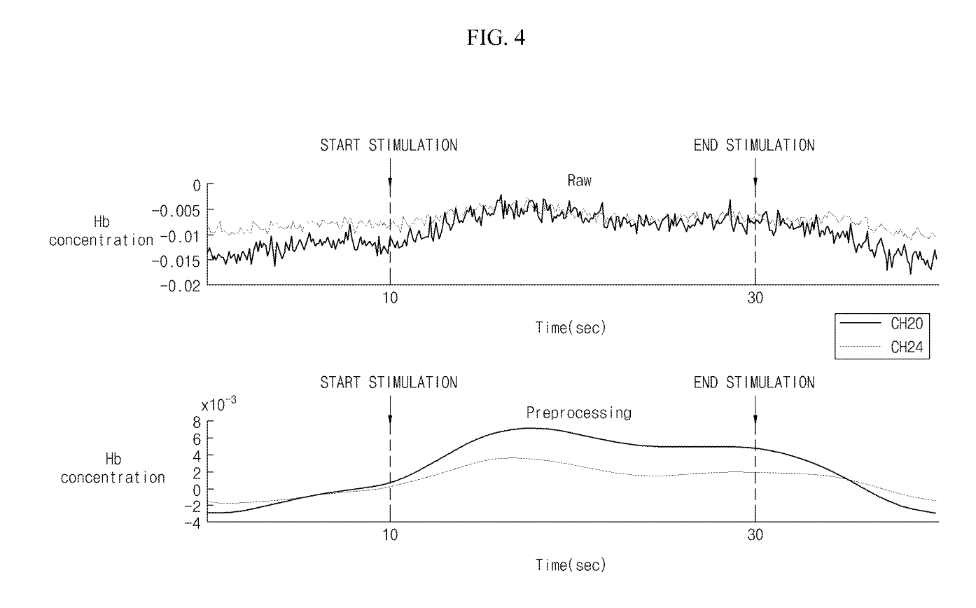

Also, when measuring the cerebral cortical blood flow and metabolism, an oxyhemoglobin concentration and a deoxyhemoglobin concentration may be measured on the basis of the blood flow as a numerical value, and noise may be removed from the measured concentration signal through a variety of preprocessing methods. FIG. 4 is a portion of an original signal and a portion of a signal with the noise removed therefrom when measuring the hemoglobin concentration.

The method extracts a feature according to a variety of learning methods using the original measured signal and the signal with the noise removed therefrom, classifies rehabilitation intention on the basis of the extracted feature, digitalizes the rehabilitation intention, and generates an instruction signal for presenting stimulation in operation S140.

The method applies functional electrostimulation to the patient to the maximum extent in terms of intensity and duration according to the digitalized instruction to operate the rehabilitation portion of the patient in operation S150.

Accordingly, the patient is allowed to actively do rehabilitation exercise by inducing the rehabilitation motivation of the patient, measuring the patient state, and providing functional electrostimulation appropriate for the patient.

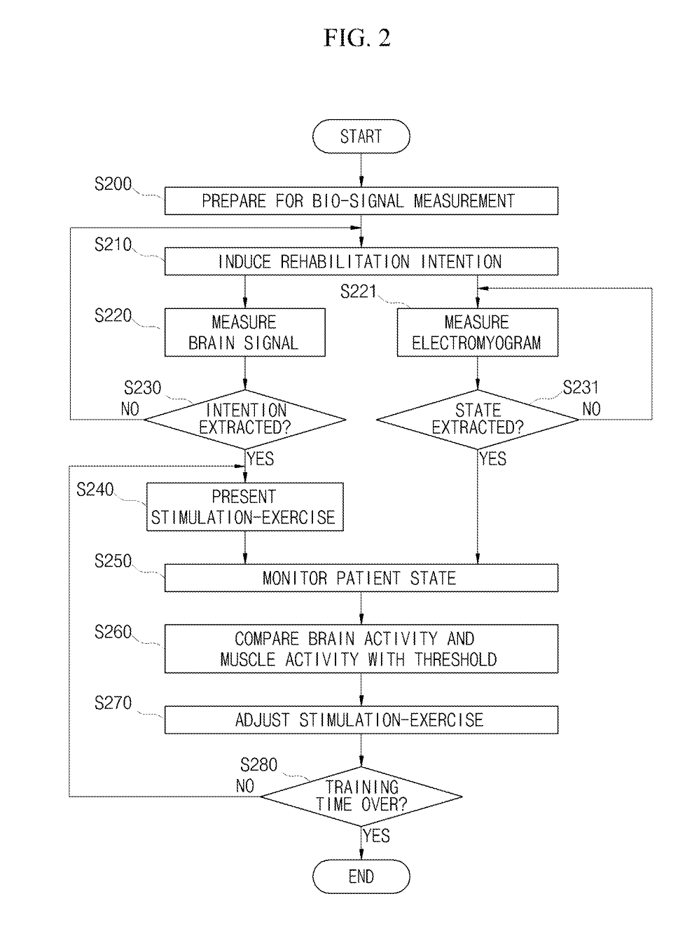

FIG. 2 is a flowchart showing a process of a self-directed rehabilitation training method according to another embodiment of the present invention, which further include attaching a patch for electromyogram measurement when attaching the patch on skin of the rehabilitation portion.

The method allows the rehabilitation patient to put on a cap for measuring a brain signal of the patient, attaches a patch to the rehabilitation portion, and attaches a patch for electromyogram measurement to a skin portion around the rehabilitation portion to prepare to measure a bio-signal in operation S200. In the same method as described with reference to FIG. 1, the method provides a rehabilitation image to the patient to induce rehabilitation intention of the patient in operation S210, measures a brain signal of the patient in operation S220, extracts the intention of the patient in operation S230, and generates an instruction for presenting stimulation appropriate for the patient, to provide the functional electrostimulation to the patient in operation S240.

Unlike the method shown in FIG. 1, the method shown in FIG. 2 further includes measuring electromyogram of the patient using the electromyogram patch attached to the patient in operation S221 and extracting a state of the patient in operation S231. The self-directed rehabilitation training method measuring the muscle activity may display rehabilitation intention recognition and muscle activity intensity on a screen to encourage a rehabilitation will of the patient.

The method monitors the state of the patient through a brain signal or electromyogram in operation S250, compares a brain activity or muscle activity with a predetermined threshold in operation S260, and adjusts intensity or duration of functional electrostimulation applied to the patient according to a result of the comparison in operation S270.

If the brain activity or muscle activity is above the threshold, the intensity or duration of the functional electrostimulation may be adjusted to be steadily reduced with time, and the functional electrostimulation may be stopped for a certain time and then provided. If the brain activity or muscle activity is significantly below the threshold, the intensity of the functional electrostimulation may be increased.

When a training time is not completed after adjusting the functional electrostimulation in operation S280, the method continuously provides the adjusted functional electrostimulation to the patient in operation S240.

The method completes the rehabilitation training if a predetermined time elapses according to a rehabilitation guideline of a doctor in operation S280, removes a variety of equipment from the patient, and waits for a certain time period until the patient is in a comfortable state. The method explains details and results of the rehabilitation training to the patient and conducts a survey about the rehabilitation training, and allows the patient to prepare for the next rehabilitation training for himself/herself.

The present invention provides allows a patient having disease on the musculoskeletal system and a patient requiring sport rehabilitation in addition to a patient having damage on the central nervous system due to brain or vertebra damage to perform active self-training with motivation and intention.

Also, the present invention allows a patient to self-directedly generate an exercise instruction of a brain and feedback sensing information to activate a sensory-motor region of the brain to enable sensory-motor looped rehabilitation, thereby enhancing neuroplasticity, musculoskeletal resilience, and exercisable range separately or simultaneously.

Furthermore, the present invention allows a patient to adjust intensity and duration of the training for himself/herself according to his/her state, thereby providing a significant effect on patient-specific flexible rehabilitation.

The above-described subject matter of the present invention is to be considered illustrative and not restrictive, and it should be understood that numerous other modifications and embodiments can be devised by those skilled in the art that will fall within the spirit and scope of the principles of the present invention. Accordingly, the embodiments of the present invention are to be considered descriptive and not restrictive of the present invention, and do not limit the scope of the present invention. The scope of the present invention should be determined by the following claims and their appropriate legal equivalents.

What is claimed is:

1. A self-directed rehabilitation training method comprising:
allowing a patient to put a cap having a measurement probe on a scalp of the patient and attaching a patch to a rehabilitation portion of the patient;
providing the patient with a rehabilitation operation screen;
measuring cerebral cortical blood flow and metabolism of the patient using the cap to generate a measured signal;
extracting a feature of the measured signal and classifying rehabilitation intention on the basis of the feature; and
converting the rehabilitation intention into an instruction for presenting stimulation and providing the patient with rehabilitation exercise according to the instruction for presenting stimulation.

2. The self-directed rehabilitation training method of claim 1, wherein the attaching of a patch to a rehabilitation portion of the patient further comprises attaching a patch for electromyogram measurement to a skin portion around the rehabilitation portion.

3. The self-directed rehabilitation training method of claim 1, wherein the providing of the patient with a rehabilitation operation screen comprises providing the patient with the rehabilitation operation screen repeatedly for a predetermined time period.

4. The self-directed rehabilitation training method of claim 1, wherein the measuring of cerebral cortical blood flow and metabolism of the patient comprises measuring the cerebral cortical blood flow and metabolism of the patient using Functional Near Infrared Spectroscopy (fNIRS) through the cap put on the scalp of the patient.

5. The self-directed rehabilitation training method of claim 1, wherein the measuring of cerebral cortical blood flow and metabolism of the patient comprises measuring the cerebral cortical blood flow and metabolism of the patient using Functional Near Infrared Spectroscopy (fNIRS) through the cap put on the scalp of the patient.

6. The self-directed rehabilitation training method of claim 1, wherein the generating of the measured signal comprises generating the measured signal by measuring an oxyhemoglobin concentration and a deoxyhemoglobin concentration on the basis of the blood flow.

7. The self-directed rehabilitation training method of claim 1, wherein the generating of the measured signal comprises removing noise from the measured signal through a preprocessing method and providing the measured signal with the noise removed therefrom when extracting the feature for the rehabilitation intention classification.

8. The self-directed rehabilitation training method of claim 1, wherein the providing of the patient with rehabilitation exercise comprises operating the rehabilitation portion of the patient by applying functional electrostimulation to the patient to the maximum extent in terms of intensity and duration according to the instruction for presenting stimulation.

9. The self-directed rehabilitation training method of claim 8, further comprising measuring a degree of muscle contraction of the patient after applying the functional electrostimulation to the patient, and if the muscle activity exceeds a predetermined value, gradually reducing the intensity and duration of the functional electrostimulation.

10. The self-directed rehabilitation training method of claim 9, further comprising providing the muscle activity to the patient through a screen.
